(19) United States
(12) Patent Application Publication
(10) Pub. No.: US 2013/0028935 A1
(43) Pub. Date: Jan. 31, 2013

(54) COMPOSITION CONTAINING HCMV PARTICLES

(71) Applicant: Leander GRODE, Braunschweig (DE)

(72) Inventor: Leander GRODE, Braunschweig (DE)

(73) Assignees: RHEIN BIOTECH GMBH, Duesseldorf (DE); VAKZINE PROJEKT MANAGEMENT GMBH, Hannover (DE)

(21) Appl. No.: 13/612,959

(22) Filed: Sep. 13, 2012

Related U.S. Application Data
(62) Division of application No. 12/599,725, filed on Sep. 9, 2010.

(30) Foreign Application Priority Data
May 11, 2007 (EP) 07009528.6

(51) Int. Cl. A61K 39/245; A61P 37/04

(52) U.S. Cl. 424/230.1

(57) ABSTRACT

The present invention is related to a composition comprising an agent selected from the group comprising HCMV virions, HCMV dense bodies and HCMV NIEP, whereby the composition is capable of elucidating an immune response while the virions, the NIEP and/or the dense bodies being non-fusiogenic.

This application is a divisional of U.S. Ser. No. 12/599,725 filed Sep. 9, 2010, which is a 35 U.S.C. 371 National Phase Entry Application from PCT/EP2008/003837, filed May 13, 2008, which claims the benefit of European Patent Application No. 07009528.6 filed on May 11, 2007, the disclosures of which are incorporated herein in their entirety by reference.

The present invention is related to a composition comprising an agent, whereby such agent is selected from the group comprising HCMV virions, HCMV dense bodies and HCMV NIEP, the use of such compositions and a method for the manufacture of such composition.

Human cytomegalovirus (HCMV), a β-herpesvirus, is a ubiquitously occurring pathogen. In an immunocompetent person, HCMV infection is normally unnoticed, having at the most mild and nonspecific symptoms. By contrast, in certain risk groups, for example in immunosuppressed patients such as AIDS patients or transplant recipients, and after prenatal infection, HCMV infection has serious manifestations.

Chemotherapeutics are available for treating HCMV infections. The success of antiviral chemotherapy of HCMV infection is restricted, however, in particular by the toxicity of the medicaments and the development of resistant variants of the virus if the duration of treatment is prolonged. In addition, the prophylactic or therapeutic use of antiviral hyperimmune sera has proved to be of only limited efficacy.

There has been work on the development of a vaccine against HCMV for many years. Thus, attempts have been made with weakened (attenuated) live vaccines to induce the desired immunity. This vaccine proved to have only limited efficacy, however. The reasons for this may be, inter alia, the restricted viability of such attenuated viruses in humans and strain-specific variations in the antigenicity. Besides the inadequacies in the induction of a permanent immunity, the use of a live vaccine must be regarded critically; lack of knowledge about the pathogenetic mechanisms in HCMV infection and the risk of reactivating the vaccine strain after immunosuppression make the use of a live vaccine appear at least questionable in these clinical situations.

In order to avoid these risks, strategies have recently been preferentially followed to develop subunit vaccines against HCMV which contain proteins from the viral envelope synthesized in various expression systems. Such envelope proteins, especially the glycoproteins gB and gH, are the essential target antigens of neutralizing antibodies against HCMV. Neutralizing antibodies are able to prevent the infection. It was possible both in experimental animals and in clinical studies to induce such neutralizing antibodies with a gB subunit vaccine. However, in humans, the antibody response induced in this way proved to be short-lived and not suitable for preventing the infection in all cases. This is detrimental to the wide use of subunit vaccines based exclusively on the gB of HCMV. The reasons which have been suggested for the limited efficacy of such antigen preparations in turn are the strain-specific variations in the immune response, lack of induction of an adequate cellular immune response, and structural restrictions of the antigen used, whose epitopes are in some cases known to be conformation-dependent.

On the basis of this experience, therefore, the requirements to be met by an effective and widely useful vaccine against HCMV are as follows: (1) Long-lasting induction of neutralizing antibodies which protect from HCMV infection in a strain-overlapping manner. This requires efficient induction of a so-called “helper cell response” (CD4-positive T lymphocytes) against HCMV to assist the maturation of antibody-secreting B lymphocytes. (2) Induction of the formation of cytotoxic T cells against HCMV. Lymphocytes of this type are of crucial importance for terminating an HCMV infection which has taken place and limiting the spread of the virus in the body. (3) Minimizing the side effects by the vaccine. The risk which might derive from an inoculated viable virus which, according to present knowledge, would have the ability to establish latency after immunosuppression cannot be estimated. The aim ought therefore to be to prepare nonviable viral antigen as vaccine.

A vaccine of that type is described in international patent application WO 00/53729.

Although this kind of vaccine is certainly helpful, high standards have to be met in terms of reducing residual infectivity of such kind of viral particles due to a possible presence of residual infectious HCMV virions. Therefore, a problem underlying the present invention was to provide for a composition containing HCMV particles and more specifically HCMV virions and/or HCMV dense bodies having no residual infectivity, particularly no residual infectivity in an assay as described herein.

A further problem underlying the present invention is to provide a species of HCMV particles which is non-infectious but provides for an antigen specific CD8+, cytotoxic T cell response. A further problem underlying the present invention is to provide a species of HCMV particles which is non-infectious but provides for an antigen specific immune response, whereby such immune response comprises antibodies against the antigen, preferably neutralizing antibodies against the antigen.

This and other problems are solved by the subject matter of the independent claims. Preferred embodiments may be taken from the dependent claims.

More specifically, the problem underlying the present invention is solved in a first aspect by a composition comprising an agent selected from the group comprising HCMV virions, HCMV dense bodies and HCMV NIEP, whereby the composition is capable of elucidating an immune response while the virions, the NIEP and/or the dense bodies being non-fusiogenic. In an embodiment, the agent is non-infectious.

The problem underlying the present invention is solved in a second aspect by a composition comprising an agent selected from the group comprising HCMV virions, HCMV dense bodies and HCMV NIEP, whereby the composition is capable of elucidating an immune response while the virions, the NIEP and/or the dense bodies being non-fusiogenic, whereby the composition is obtainable by a process comprising the steps of:
a) providing one or several of the agents;
b) treating the agent(s) to render them non-fusiogenic while still being capable of inducing an immune response.

In an embodiment, the agent is non-infectious.

In an embodiment of the first and second aspect the immune response is an antigen specific CD8+ response.

In an embodiment of the first and second aspect the immune response is an antigen specific cytotoxic T cell response.

In an embodiment of the first and second aspect the immune response is an antigen specific CD8+, cytotoxic T cell response

In an embodiment of the first and second aspect the immune response is an antigen specific antibody response, preferably the immune response is an antigen specific antibody response, whereby the antibodies are neutralizing antibodies.

In an embodiment of the first and second aspect the immune response is an antigen specific CD4+ T helper cell response.

In an embodiment of the first and second aspect the antigen is a HCMV antigen, whereby the HCMV antigen is preferably selected from the group comprising pp65 antigen, pp65 antigen derivatives, pp28 and pp28 derivatives, pp150 and pp150 derivatives, gB and gB derivatives, gH and gH derivatives, and immediate early antigens and derivatives thereof, glycoproteins and glycoprotein derivatives, preferably HCMV glycoproteins and HCMV glycoprotein derivatives, whereby the glycoproteins are preferably gM and gM derivatives, or gN and gN derivatives.

Within the group of the immediate early antigens the immediate early antigen-1 (IE-1) is particularly preferred.

In an embodiment of the first and second aspect the agent is inactivated.

In an embodiment of the first and second aspect the composition is a pharmaceutical composition or a diagnostic composition.

The problem underlying the present invention is solved in a third aspect by the use of an agent, whereby the agent is selected from the group comprising an HCMV virion, HMCV dense body and HCMV NIEP, or of a composition comprising such agent, whereby the agent is non-fusiogenic, for the manufacture of a medicament, preferably for the elucidation of an immune response against one or several of the antigens of HCMV.

The problem underlying the present invention is solved in a fourth aspect by the use of an agent, whereby the agent is selected from the group comprising an HCMV virion, HCMV dense body and HCMV NIEP, or of a composition comprising such agent, preferably a composition as defined in the third aspect, whereby the composition and/or the agent has been subjected to inactivation, for the manufacture of a medicament for the elucidation of an immune response, preferably an immune response against one or several of the antigens of HCMV.

The problem underlying the present invention is solved in a fifth aspect by the use of an agent, whereby the agent is selected from the group comprising an HCMV virion, HCMV dense body and HCMV NIEP, or of a composition comprising such agent, whereby the agent is non-fusiogenic, for the manufacture of a vaccine.

The problem underlying the present invention is solved in a sixth aspect by the use of an agent, whereby the agent is selected from the group comprising an HCMV virion, HCMV dense body and HCMV NIEP, or of a composition comprising such agent, preferably a composition as defined in the third aspect, whereby the composition and/or the agent has been subjected to inactivation, for the manufacture of a vaccine.

In an embodiment of the third and fourth aspect the immune response is an antigen specific CD8+ response.

In an embodiment of the third to sixth aspect the immune response is an antigen specific cytotoxic T cell response.

In an embodiment of the third to sixth aspect the immune response is an antigen specific CD8+, cytotoxic T cell response.

In an embodiment of the third to sixth aspect the immune response is an antigen specific antibody response, preferably the immune response is an antigen specific antibody response, whereby the antibodies are neutralizing antibodies.

In an embodiment of the third to sixth aspect the immune response is an antigen specific CD4+ T helper cell response.

In an embodiment of the third to sixth aspect the antigen is a HCMV antigen, whereby the HCMV antigen is preferably selected from the group comprising pp65 antigen, pp65 antigen derivatives, pp28 and pp28 derivatives, pp150 and pp150 derivatives, gB and gB derivatives, gH and gH derivatives, and immediate early antigens and derivatives thereof, glycoproteins and glycoprotein derivatives, preferably HCMV glycoproteins and HCMV glycoprotein derivatives, whereby the glycoproteins are preferably gM and gM derivatives, or gN and gN derivatives.

In an embodiment of the fifth and sixth aspect the vaccine is for the treatment and/or prevention of HCMV infection.

In an embodiment of the fifth and sixth aspect the vaccine is for the treatment and/or prevention of a disease caused by HCMV in a transplant donor and/or transplant recipient.

The problem underlying the present invention is solved in a seventh aspect by the use of an agent, whereby the agent is selected from the group comprising an HCMV virion, HMCV dense body and HCMV NIEP, or of a composition comprising such agent, whereby the agent is non-fusiogenic, for the manufacture of a diagnostic agent.

The problem underlying the present invention is solved in an eighth aspect by the use of an agent, whereby the agent is selected from the group comprising an HCMV virion, HMCV dense body and HCMV NIEP, or of a composition comprising such agent, whereby the composition and/or the agent has been subjected to inactivation, for the manufacture of a diagnostic agent.

The problem underlying the present invention is solved in a ninth aspect by a method for the manufacture of a composition as defined in the first and second aspect, comprising the steps of
a) providing an agent selected from the group comprising HCMV virions, HCMV NIEP and HCMV dense bodies;
b) treating the agent to render it non-fusiogenic while said agent still being capable of inducing an immune response.

In an embodiment of the ninth aspect the treatment of step b) is one or any combination of measures selected from the group comprising UV treatment, high energy irradiation, low pH treatment, heat treatment and treatment with cross-linking agents.

In a preferred embodiment of the ninth aspect the UV treatment is UVC treatment, whereby the wavelength is about 100 nm-280 nm, or treatment with long wave UV.

It is within the present invention that rather than using UV C for inactivation, also long wave UV may be used. In such case, long wave UV is used together with a photoreactive agent which is activated by said long wave UV. In an embodiment such photoreactive agent is amotosalen which is used together with UVA, 4′-aminomethyl-4,5′,8-trimethylpsoralen which is used together with UV A, and dimethylmethylene blue which is used together with UV A and UV B, respectively.

In an embodiment of the ninth aspect the UV treatment is using a dose range from about 100 to about 2000 mJ/cm²′ preferably from about 100 to about 1000 mJ/cm² and more preferably from about 150 to about 900 mJ/cm².

In an embodiment of the ninth aspect prior to, concomitantly with or subsequently to the UV treatment the agent is subject to gamma irradiation.

In a preferred embodiment of the ninth aspect the high energy irradiation is gamma irradiation.

In a further preferred embodiment of the ninth aspect the gamma radiation in connection with the gamma irradiation is administered within a dosage range from about 15 to about 70 KGy, more preferably about 20 to about 65 KGy and more preferably about 20 to about 60 KGy.

In an embodiment of the ninth aspect the treatment is low pH treatment and the low pH treatment comprises exposure of the agent to a pH of about 0 to 5, preferably 1 to 4.5 and more preferably 2 to 4.5.

In a preferred embodiment of the ninth aspect the agent is subject to the low pH treatment for about 0.5 to 24 hours, preferably about 0.5 to 12 hours and more preferably about 0.5 to 6 hours.

In an embodiment of the ninth aspect the agent is subject to the low pH treatment at about 1 to 50° C., preferably at about 1 to about 45° C. and more preferably at about 1 to about 40° C.

In an embodiment of the ninth aspect the heat treatment comprises the incubation of the agent at a temperature between about 37.5° C. and about 65° C., preferably at a temperature between about 37.5 and about 60° C. and more preferably at a temperature between about 37.5 and about 56° C.

In a preferred embodiment of the ninth aspect the agent is incubated for a period between about 5 seconds and about 36 hours, preferably between about 5 seconds and about 30 hours, and more preferably between about 5 seconds and about 24 hours.

In an embodiment of the ninth aspect the treatment is treatment with one or several cross-linking agent and whereby the cross-linking agent is each and independently selected from the group comprising lactones, ethoxides and aldehydes.

In an embodiment of the ninth aspect the cross-linking agent is B-propio lactone.

In an embodiment of the ninth aspect the cross-linking agent is ethylene oxide.

In an embodiment of the ninth aspect the cross-linking agent is formaldehyde.

In an embodiment of the ninth aspect the agent is exposed to the cross-linking agent with the concentration of the cross-linking agent and more preferably of B-propiolactone in the medium containing the agent being between about 0.05 and about 10% (v/v), preferably between about 0.05 and about 10% (v/v), and more preferably between about 0.05 and about 7.5% (v/v).

In an embodiment of the ninth aspect the agent is incubated with the cross-linking agent and preferably B-propio lactone for a period between about 1 minute and about 72 hours, preferably between about 1 minute and about 48 hours, and more preferably between about 1 minute and about 24 hours.

In an embodiment of the ninth aspect the agent is incubated at a temperature between about 1° C. and about 60° C., preferably at a temperature between about 1° C. and about 50° C., more preferably at a temperature between about 1° C. and about 40° C.

According to the prior art it is thought that HCMV particles, in order to generate an antigen specific CD8+, cytotoxic T-cell response to viral antigens and virus infected cells, have to fuse with target cell membrane (Pepper) et al 2000 J Virol 74:6132-6146). This fusion is deemed a prerequisite in order to channel viral antigens into the MHC class I pathway of antigen processing and presentation, either directly after entry into cells or subsequent to intracellular transcription and translation of viral proteins (Pepper) et al 2000 J Virol 74:6132-6146). Antigen presentation via the MHC class I pathway in turn is a prerequisite for the immune system to be able to mount or elucidate an antigen specific CD8+, cytotoxic T-cell response to viral antigens and virus infected cells.

The present inventors have now found that HCMV particles which are no longer fusiogenic are still capable of eliciting such kind of antigen specific CD8+, cytotoxic T-cell response to viral antigens and virus infected cells. This applies particularly to HCMV particles which have been or are treated by or generated by applying one or several of the means and methods, respectively, described herein for or in connection with any inactivation and inactivation procedure, respectively. This first finding of the present invention is insofar surprising as according to the prior art such fusiogenicity of HCMV particles was thought to be a prerequisite for this kind of T-cell response as outlined above.

Such non-fusiogenicity can be generated by applying to HCMV particles measures and methods, in essence, conventionally known in the art to inactivate HCMV or reduce infectivity of HCMV and HCMV particles, preferably those measures and methods described herein which are also referred to as means and methods, respectively, described herein for or in connection with inactivation and inactivation procedures. These inactivating or inactivation measures and methods, respectively, are used and performed to the extent that said non-fusiogenicity is imposed upon or imparted to such HCMV particles.

The second finding underlying the present invention is related to providing HCMV particles or compositions comprising such HCMV particles, having no infectivity, preferably no residual infectivity, whereby said HCMV particles and compositions comprising the same, are generated by applying any of the measures and methods, respectively, disclosed herein in connection with the first aspect of the present invention, starting from HCMV particles or HCMV particles containing compositions still containing infectious HCMV particles and in particular virions of HCMV. Also this kind of HCMV particles has surprisingly been found to be suitable to elicit an immune response as described herein, i.e. in particular an antigen specific CD8+ cytotoxic T cell response, an antigen specific antibody response, whereby the antibody response is preferably a response providing for antigen specific neutralizing antibodies, and an antigen specific CD4+ T helper cell response.

It is generally known among the ones skilled in the art, that induction of antigen-specific antibody responses and antigen-specific CD8+ cytotoxic T cell responses, respectively, requires generation of antigen-specific CD4+ T helper cells. Therefore, in the present invention the generation of antigen-specific CD4+ T helper cells is evidenced by demonstrating pp65-specific CD8+ cytotoxic T cell responses and by generation of antibodies, in particular neutralizing antibodies, specific to HCMV antigens. Hence, HCMV particles that received treatment to inactivate residual HCMV infectivity and to render the material non-fusiogenic are still able to induce antigen-specific CD4+ T helper cells.

As preferably used herein, the term no residual infectivity means that no infectious particle and in particular no infectious HCMV particle can be detected in a sample or composition comprising HCMV particles, using an infectivity assay, more preferably the infectivity assay as described herein. In other words, a composition or preparation containing one or several of HCMV dense bodies, HCMV virions and/or HCMV NIEP, is a composition and preparation, respectively, in which no infectious HCMV or HCMV particle may be detected in the very infectivity assay. It will be acknowledged by the person skilled in the art that the detection limit will define whether and if so to which extent, i.e. how many of such HCMV particles are nevertheless present in the respective composition and preparation, respectively.

As preferably used herein the term HCMV particles comprises HCMV virions, HCMV dense bodies and HCMV NIEP.

As preferably used herein HCMV virions are infectious viral particles that consist of a membrane, the tegument and a capsid that contains viral DNA.

As preferably used herein HCMV dense bodies (DB) are non-infectious HCMV particles that lack the HCMV capsid and the HCMV DNA, but comprise a membrane and the tegument.

As preferably used herein HCMV NIEP are non-infectious, enveloped HCMV particles that lack DNA, but comprise a membrane, a capsid and the tegument.

As preferably used herein, the term dense bodies comprises both non-recombinant and recombinant dense bodies. Recombinant dense bodies preferably express one or several heterologous antigens.

As preferably used herein the term NIEP comprises both non-recombinant and recombinant NIEP. Recombinant NIEP preferably express one or several heterologous antigens.

As preferably used herein the term virions comprise both non-recombinant and recombinant virions. Recombinant virions preferably express one or several heterologous antigens.

As preferably used herein, the term heterologous antigen is an antigen which is expressed in a different expression context. In one embodiment, such different expression context is a context where the antigen is an antigen which is not inherent to the respective wildtype dense bodies, wildtype NIEP and wildtype virion. More specifically, the heterologous antigen is preferably an antigen which is either an internal or inner constituent of a HCMV particle, including but not limited to a non-structural HCMV antigen, or an antigen of a heterologous organism, preferably a heterologous pathogen. In a further embodiment such different context is a context which differs from the wildtype context in that the promoter which controls the expression of the antigen is different from the promoter controlling the expression of the antigen in wildtype dense bodies, wildtype NIEP and wildtype virions. In a further embodiment the different context consists of a different translational system or translational background where the antigen is expressed, which, again, is different from the wildtype system or wildtype background. More specifically, the wildtype dense bodies are HCMV wildtype dense bodies, the wildtype NIEP are wildtype HCMV NIEP and the wildtype virions are wildtype HCMV virions.

As preferably used herein, wildtype means a strain or from a strain which is capable of forming dense bodies, preferably under in vitro cell culture conditions. Such wildtype or wildtype strain is preferably Ad169 and Towne.

It will be acknowledged that any statement, embodiment, feature or advantage described herein in connection with the HCMV particles is also and in particular applicable to HCMV dense bodies and HCMV NIEP and more particularly applicable to HCMV dense bodies.

As preferably used herein the term fusiogenicity designates the ability of viral and subviral particles such as of HCMV to fuse to target cells mediated by fusion of the viral and subviral particle membrane, respectively, with the cellular membrane of a target cell. Such viral and subviral particles, respectively, are referred to as “fusiogenic”; whereas such viral and subviral particles, respectively, which are not able to fuse to target cells are referred to as non-fusiogenic.

Based upon the finding of the present inventors, namely that such HCMV particles including, but not limited to, HCMV virions, HCMV dense bodies and HCMV NIEP, which are or have been treated to inactivate residual HCMV infectivity thereof, are still able to efficiently induce an antigen specific CD8+, cytotoxic T-cell response to viral antigens despite a proven loss of fusiogenicity, means and methods for inactivating HCMV infectivity and more specifically inactivating residual HCMV infectivity by means and under conditions, respectively, which go along with loss of fusiogenicity can be applied to HCMV particle containing preparations and compositions, respectively, to generate both a safe HCMV vaccine and an antigen specific CD8+, cytotoxic T-cell response.

The HCMV particles which form the starting material for the generation of the HCMV particles and compositions comprising the same, respectively, each as subject to the present invention, are preferably viral particles which are released after infection of mammalian cells by HCMV. Such HCMV particles which are used as a starting material are surrounded by a lipid membrane which makes it possible to fuse the particles to certain mammalian cells so that their contents enter the cytoplasm of the cells, although, in accordance with the first aspect of the present invention, such particles obtained upon subjecting them to the means and methods, respectively, described herein in connection with or suitable for inactivation, are non-fusiogenic. Irrespective of this, the membrane of the HCMV particles contains viral glycoproteins which represent the main antigens for virus-neutralizing antibodies. In addition, they contain the viral antigen pp65 (ppUL83) which is a very immunogenic target for T-helper cells and is an essential antigen for inducing cytotoxic T lymphocytes (CTL) against HCMV.

The kind of immune response created by the HCMV particles and in particular the HCMV virions, HCMV NIEP and/or HCMV dense bodies, and the compositions comprising the same, respectively, each in accordance with the present invention, is, irrespective of the route of administration a T-helper cell response of the Th1 type. Because of this characteristic, the HCMV particles and in particular the HCMV virions, HCMV dense bodies and/or the HCMV NIEP, and the compositions comprising at least one of them, respectively, are suitable as vaccines against HCMV.

It will be acknowledged by the person skilled in the art that the HCMV particles of the present invention and any composition comprising the same may be used for the preparation of a medicament which is used for the treatment and/or prevention of a disease, preferably such disease is one which involves HCMV as a causative or opportunistic agent.

In a preferred embodiment the medicament is a vaccine.

A particular group of subjects, preferably mammalian subjects and more preferably human subjects, for the treatment and/or prevention of which such medicament is used in accordance with the present invention are those which suffer from or are at risk to develop a disease caused by HCMV, whereby such subjects are transplant donors and/or transplant recipients. It will also be acknowledged by the person skilled in the art that the HCMV particles of the present and any composition comprising the same may be used for the preparation of a diagnostic agent. More preferably such diagnostic is one for the diagnosis of a disease which involves HCMV as a causative or opportunistic agent.

Finally the HCMV particles may be used for the generation or in the manufacture of a medicament or of a diagnostic, whereby the medicament and diagnostic agent are one of an agent selected from the group comprising antibodies, aptamers and spiegelmers, whereby the agent is directed, preferably specifically directed against one or several of the HCMV particles of the present invention. The generation of such antibodies, aptamers and spiegelmers are known to the person skilled in the art.

The manufacture of an antibody specific for the HCMV particles of the present invention is known to the one skilled in the art and, for example, described in Harlow, E., and Lane, D., “Antibodies: A Laboratory Manual,” Cold Spring Harbor Laboratory, Cold Spring Harbor, N.Y., (1988). Preferably, monoclonal antibodies may be used in connection with the present invention which may be manufactured according to the protocol of Cesar and Milstein and further developments based thereon. Antibodies as used herein, include, but are not limited to, complete antibodies, antibody fragments or derivatives such as Fab fragments, Fc fragments and single-stranded antibodies, as long as they are suitable and capable of binding to HCMV particles of the present invention. Apart from monoclonal antibodies also polyclonal antibodies may be used and/or generated. The generation of polyclonal antibodies is also known to the one skilled in the art and, for example, described in Harlow, E., and Lane, D., “Antibodies: A Laboratory Manual,” Cold Spring Harbor Laboratory, Cold Spring Harbor, N.Y., (1988). Preferably, the antibodies used for therapeutical purposes are humanized or human antibodies as defined above.

The antibodies which may be used according to the present invention may have one or several markers or labels. Such markers or labels may be useful to detect the antibody either in its diagnostic application or its therapeutic application. Preferably the markers and labels are selected from the group comprising avidine, streptavidine, biotin, gold and fluorescein and used, e.g., in ELISA methods. These and further markers as well as methods are, e.g. described in Harlow, E., and Lane, D., “Antibodies: A Laboratory Manual,” Cold Spring Harbor Laboratory, Cold Spring Harbor, N.Y., (1988).

It is also within the present invention that the label or marker exhibits an additional function apart from detection, such as interaction with other molecules. Such interaction may be, e.g., specific interaction with other compounds. These other compounds may either be those inherent to the system where the antibody is used such as the human or animal body or the sample which is analysed by using the respective antibody. Appropriate markers may, for example, be biotin or fluoresceine with the specific interaction partners thereof such as avidine and streptavidine and the like being present on the respective compound or structure to interact with the thus marked or labelled antibody.

Aptamers as subject to the present invention are D-nucleic acids which are either single stranded or double stranded and which specifically interact with a target molecule. The manufacture or selection of aptamers is, e.g., described in European patent EP 0 533 838. Basically the following steps are realized. First, a mixture of nucleic acids, i.e. potential aptamers, is provided whereby each nucleic acid typically comprises a segment of several, preferably at least eight subsequent randomised nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic acid is then optionally selected. These specifically binding nucleic acid(s) are referred to aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agens. However, it is also within the present invention that the thus selected or generated aptamers may be used for target validation and/or as lead substance for the development of medicaments, preferably of medicaments based on small molecules. This is actually done by a competition assay whereby the specific interaction between the target molecule and the aptamer is inhibited by a candidate drug whereby upon replacement of the aptamer from the complex of target and aptamer it may be assumed that the respective drug candidate allows a specific inhibition of the interaction between target and aptamer, and if the interaction is specific, said candidate drug will, at least in principle, be suitable to block the target and thus decrease its biological availability or activity in a respective system comprising such target.

The thus obtained small molecule may then be subject to further derivatisation and modification to optimise its physical, chemical, biological and/or medical characteristics such as toxicity, specificity, biodegradability and bioavailability.

The generation or manufacture of spiegelmers which may be used or generated according to the present invention using the HCMV particles of the present invention is based on a similar principle. The manufacture of spiegelmers is described in the international patent application WO 98/08856. Spiegelmers are L-nucleic acids, which means that they are composed of L-nucleotides rather than aptamers which are composed of D-nucleotides as aptamers are. Spiegelmers are characterized by the fact that they have a very high stability in biological system and, comparable to aptamers, specifically interact with the target molecule against which they are directed. In the purpose of generating spiegelmers, a heterogonous population of D-nucleic acids is created and this population is contacted with the optical antipode of the target molecule, in the present case for example with the D-enantiomer of the naturally occurring L-enantiomer of the HCMV particles of the present invention or wart thereof. Subsequently, those D-nucleic acids are separated which do not interact with the optical antipode of the target molecule. However, those D-nucleic acids interacting with the optical antipode of the target molecule are separated, optionally determined and/or sequenced and subsequently the corresponding L-nucleic acids are synthesized based on the nucleic acid sequence information obtained from the D-nucleic acids. These L-nucleic acids which are identical in terms of sequence with the aforementioned D-nucleic acids interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

It is within the present invention that such medicament and diagnostic may be used for the treatment, prevention and diagnosis, respectively, of each and any of the diseases and conditions recited herein in connection with the use of the HCMV particles of the present invention. It will be understood by the person skilled in the art that the HCMV particles of the present invention may also be used as medicaments and vaccines for the treatment and/or prevention of those diseases which are treatable by eliciting an immune response against an antigen and said antigen being expressed by the HCMV particles of the present invention. It is within the present invention that such antigen is homologous or heterologous, in particular relative to said HCMV particle.

Methods to determine whether a HCMV particle and in particular the HCMV virion and/or HCMV dense body and a composition containing at least one of them, respectively, is capable of elucidating an immune response, while the particles, virions and/or the dense bodies being preferably non-fusiogenic are evident to persons skilled in the art, at least in the light of the present invention. Some of the respective tests are described herein, in particular in the example section hereof.

In order to provide for the immune response the HCMV particles of the present invention must contain or provide the relevant antigens for inducing antigen specific antibodies, preferably neutralising antibodies and for stimulating helper cells (T_{H}-lymphocytes) and cytotoxic T-cells (CTL).

In principle, HCMV antigens which are suitable to elicit an immune response, preferably in man, are in particular the following one, although a person skilled in the art will acknowledge that other HCMV antigens may also be active insofar (Sylwester A W, JEM Vol. 202, Sep. 5, 2005, p. 673-685).

HCMV antigens recognized by CD4+ T cells are preferably selected from the group comprising UL55 (gB), UL83 (pp65), UL86, UL99 (pp28), UL122 (IE2), UL36, UL48, UL32 (pp150), UL113, IRS-1, UL123 (IE1), UL25, UL141, UL52, UL82 (pp71), US22, UL75 (gH), US23, UL69, US26, UL44 (pp50), UL16, US3, US18, UL78, UL18, UL17, TRL14, UL100, UL45, UL145, UL154, UL43, UL152, UL144, UL24, UL4 (gp48), UL49, UL102 and UL87. More preferably, antigens subject to CD4+ T cell responses are selected from the group comprising UL55, UL83, UL86, UL99, UL153 and UL32.

HCMV antigens recognized by CD8+ T cells are preferably selected from the group comprising UL48, UL83 (pp65), UL123 (IE1), UL122 (IE2), US32, UL28, US29, US3, UL32 (pp150), UL55 (gB), UL94, UL69, UL105, UL82 (pp71), UL99 (pp28), UL154, UL44 (pp50), UL86, UL33, UL49, US1, UL150, UL34, US30, TRL14, IRS-1, UL36, UL37, UL75 (gH), UL45, UL153, UL116 and UL54. More preferably, antigens subject to CD8+ T cell responses are selected from the group comprising UL123, UL83, UL122, UL28, UL48, US3, UL151, UL82, UL94, US29, UL99, UL103, US32, US24 and UL36.

Neutralizing antibodies are, according to the present state of knowledge, after HCMV infection formed exclusively against viral envelope proteins, and especially against the glycoproteins gB, gH, gM and gN (Shen et al, Vaccine 20, 2007).

T_{H} cells are formed mainly against tegument proteins of the virus, and particularly against the so-called pp65 (ppUL83), gH and gB (Sylwester et al., J. Exp. Medicine Vol. 202, 2005). More specifically, pp65 is an essential antigen for the induction of CTL against HCMV. Presentation of pp65 takes place not only as usual after de novo synthesis by cells in connection with MHC class I molecules; it can also be introduced into the MHC I presentation pathway by so-called “exogenous loading”.

Said antigens are the essential constituents of the HCMV particles of the present invention and more specifically of the HCMV dense bodies and the HCMV NIEP, each of the present invention. Most specifically the dense bodies (DB) are structures which are visible under the electron microscope. The most abundant protein (mass) in DB is the tegument protein pp65. DB are comparable with virus particles in being provided with a cellular lipid membrane which is also reverted to as the envelope, modified by viral glycoproteins. The viral glycoproteins are very probably in the natural conformation in this envelope. Since DB contain no viral DNA and no viral capsid, they are non-infectious. They can be concentrated in large quantity from the cell culture supernatant by established methods.

The following embodiments are described by reference to pp65. However, it will be understood that, in principle, the same considerations are applicable to other antigens suitable for the practicing of the present invention and/or the antigens described herein either directly or by reference.

In a further embodiment, HCMV particles which contain a fusion protein which comprises in one part one or more sections of the viral T-cell antigen pp65 (ppUL83) or the complete protein and in another part one or more sections of one or more other proteins are disclosed and described.

This makes it possible to optimize the antigenicity of the HCMV particles because this fusion protein is present in large quantity in the particles. It is additionally known that expression of antigens of the cellular and humoral immune response in one molecule can distinctly increase the antigenicity. The various sections of pp65 and the other proteins can be fused together directly but it is also possible for example for linker sequences, which are not a natural constituent of one of the proteins involved, to be present between the various sections. Sequences of this type may arise because of the cloning or be introduced deliberately in order to influence the properties of the antigen. However, the fusion protein preferably contains no foreign sequences which are not a constituent of one of the fusion partners. In such embodiments, the fusion protein consists of one or more parts of pp65 and one or more parts of one or more other proteins.

It applies to all the embodiments mentioned hereinafter that the complete pp65 or one or more parts thereof can be present in the fusion protein. The statement “a fusion protein (consisting) of pp65” is not for the purposes of this application to be understood as restricted to complete pp65. A “part” or “section” of a protein present in the fusion protein comprises at least 6, preferably at least 8, most preferably at least 9, 15 or 20 consecutive amino acids of the protein from which it is derived.

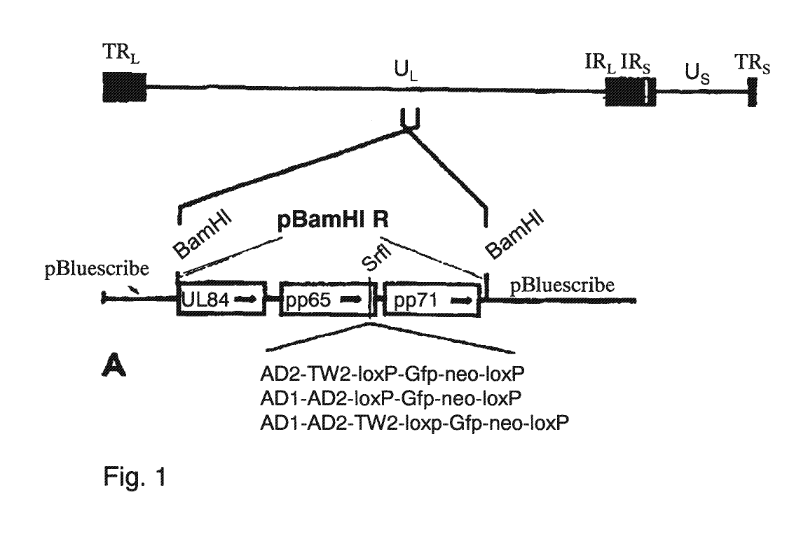

A preferred embodiment comprises a fusion protein of pp65 (ppUL83) and one or more neutralizing epitopes of the viral glycoproteins gB or gH. Particles of this type can be generated as depicted in FIG. 1. The fusion protein can enter, via antigen-specific uptake, glycoprotein-specific B cells which in turn are able to present the epitopes both of the glycoproteins and of pp65 in the context of MHC class II. In addition, it is also possible for portions of the fusion protein to be presented by professional antigen-presenting cells (APC) in the context of MHC class II. In both cases the result is efficient stimulation of the T_{H} response both to the pp65 and to viral glycoproteins. These CD4 positive T_{H} cells are able to stimulate glycoprotein-specific B cells, which present peptides of pp65 and viral glycoproteins in the context of MHC class II, to form antibodies, in particular neutralizing antibodies, whereby the antibodies are directed in one embodiment to a homologous antigen, and in another embodiment to a heterologous antigen. In addition, particles of this type can, like infectious virions, be taken up into cells, and peptides of pp65 can be introduced by exogenous loading into the MHC class I pathway. This achieves, unusually for dead vaccines, a stimulation of the CTL response to HCMV.

In a further preferred embodiment, the HCMV particles contain a fusion protein consisting of pp65 and one or more parts of another protein of HCMV, the IE1 protein (ppUL123). The parts of the IE1 protein which are to be present in particular are those against which cytotoxic T cells are formed in humans during natural infection. Peptides of the IE1 protein are in some cases presented by different MHC class I molecules than are peptides of pp65. The addition of such further “CTL epitopes” from IE1 is intended to ensure that, after immunization, inoculated subjects who express different MHC class I molecules are able to generate CTL against HCMV in as comprehensive a manner as possible.

In a further preferred embodiment, the HCMV particles contain a fusion protein consisting of pp65, of one or more neutralizing epitopes of HCMV glycoproteins and of one or more CTL epitopes of IE1. Fusion of pp65 with neutralizing epitopes and CTL epitopes is intended to ensure that it is possible simultaneously for both neutralizing antibodies and CTL to be formed by inoculated subjects in as comprehensive a manner as possible, i.e. by the maximum number of people differing in MHC class I pattern.

In a further preferred embodiment, the HCMV particles contain a fusion protein of pp65 and one or more epitopes of another human pathogen. Suitable portions of other human pathogens are antigens against which neutralizing antibodies are formed in humans. It is possible through fusion of such “neutralizing antigens” with the T-cell antigen pp65 to expect a marked increase in the immune response, i.e. an antibody response, compared with the use of the isolated “neutralizing antigen”. Examples of such “neutralizing antigens” which should be mentioned are surface proteins of hepatitis B virus (from the HBsAG region), of hepatitis C virus (for example E2), of human immunodeficiency viruses (HIV, from the Env region), of influenza virus (hemagglutinin, neuraminidase, nucleoprotein) or other viral pathogens. Further suitable human pathogens are bacteria such as Haemophilus influenzae, Bordetella pertussis, Mycobacterium tuberculosis, Neisseria meningitidis and others. Finally, antigens from eukaryotic pathogens such as plasmodia (malaria) could be fused to pp65. Such antigens or fusion proteins are also referred to herein as antigen derivatives and a fusion protein acting as an antigen comprising a full length or particle pp65 is also referred to herein as pp65 antigen derivative.

In a further preferred embodiment, the HCMV particles contain a fusion protein consisting of pp65 and one or more portions of proteins or peptides, whereby the pp65 acts may act as aq scaffold for said proteins and peptides, respectively, of other pathogens against which CTL are generated in humans on natural infection with these pathogens. Examples of such CTL epitopes which may be mentioned are portions of proteins of HIV-1, of HBV, of HCV or of influenza virus. The intention of such a procedure is to utilize the unique immunogenic properties of DB for generating protective CTL, i.e. cytotoxic T lymphocytes, preferably CD8+ cytotoxic T cells, against heterologous pathogens in humans.

In a further preferred embodiment, the HCMV particles contain a fusion protein consisting of pp65, of one or more neutralizing epitopes of a heterologous pathogen and of one or more CTL epitopes of the same pathogen. This fusion is intended to ensure that inoculated subjects are able to form both protective antibodies and CTL against this pathogen.

The invention additionally relates to HCMV particles containing at least two different glycoproteins which are variants of the same glycoprotein from different HCMV strains.

A preferred embodiment contains exactly two variants, one variant corresponding to the HCMV Towne strain, and the other variant corresponding to the HCMV Ad169 strain. The preferred embodiment contains the glycoprotein gB both of the Towne strain and of the Ad169 strain.

These two proteins can be incorporated with identical efficiency into the membrane of recombinant dense bodies in the infected cell. Such recombinant dense bodies are suitable for inducing not only the strain-overlapping but also the strain-specific neutralizing immune response to the two prototype HCMV strains.

It is within the present invention that apart from wildtype antigens, i.e. such as present in HCMV wildtype strains, or non-recombinant antigens, derivatives thereof can be used in the practicing of the present invention. The term derivative as used herein in connection with an antigen preferably refers to an antigen which is a recombinant antigen. A recombinant antigen is an antigen, in an embodiment, which is compared to the full length antigen truncated, comprises one or several amino acid changes when having the same length as the wildtype antigens, or comprise additional amino acid residues. It is within the present invention that the additional amino acid residues may be added to the truncated form of the antigen or the form which comprises one or several amino acid changes. Such truncation can be performed to an extent such that the antigen characteristic of such truncated antigen is still existing. In another embodiment, the recombinant antigen comprises apart from the full length antigen or the truncated antigen a further moiety. Such further moiety is preferably derived from an antigen of a virus, whereby such virus is different from HCMV, an antigen of a microorganism, preferably a pathogenic microorganism, or an antigen of a non-microorganism which is a pathogen. Preferably the pathogenic microorganism is a microorganism pathogenic to mammals and more specifically to humans, and the pathogen is a pathogen which is pathogenic to mammals and more specifically to humans. The further moiety can be the full length antigen or a truncated from thereof. In an embodiment the further moiety is suitable to elicit one or several of the immune response described herein. In a further embodiment the derivative of a/the antigen is a heterologous antigen. In a still further embodiment the derivative of a/the antigen is a heterologous antigen as preferably defined herein. Preferably the immune response which can be elicited by the HCMV particles in their diverse forms is at least one out of the followings: antigen specific CD8+ T cell response, antigen specific cytotoxic T cell response, antigen specific CD8+ cytotoxic T cell response, antigen specific antibody response, whereby, preferably, the antibodies of such antibody response are neutralizing antibodies, antigen specific CD4+ T helper cell response.

The virions and/or dense bodies of HCMV particularly useful in the practising of the present invention can be prepared as described in international patent application WO 00/53729.

It will be acknowledged by a person skilled in the art that the various measures described herein for inactivation are, as such, known in the art and can be applied to the present invention.

The present invention is now further illustrated by the figures and the examples from which further features, embodiments and advantages may be taken, whereby

FIG. 1 shows the strategy for generating recombinant DB which contain fusion protein with homologous or heterologous antigens;

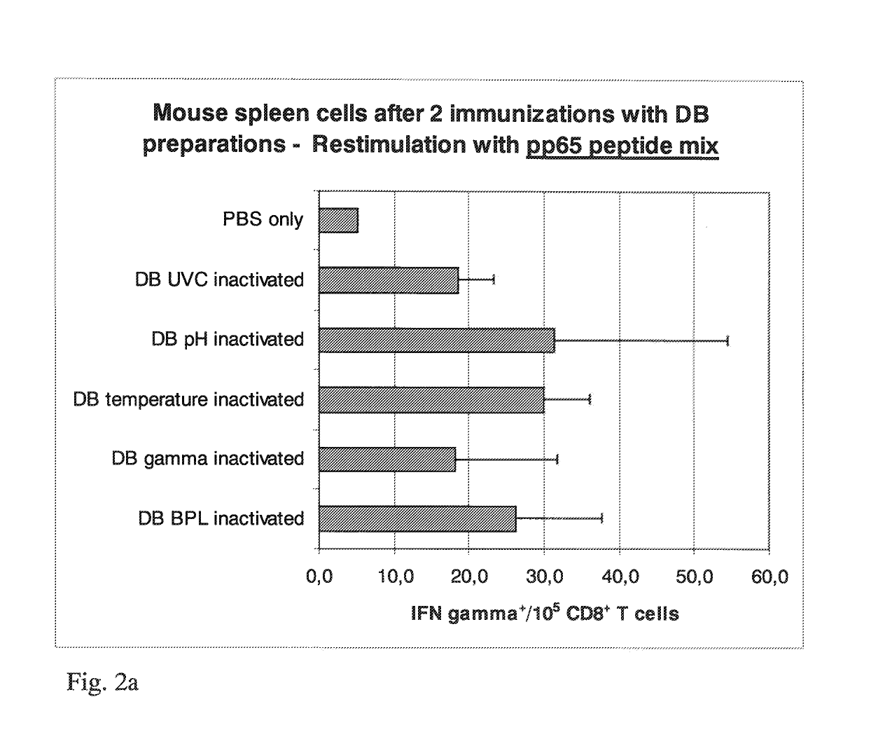

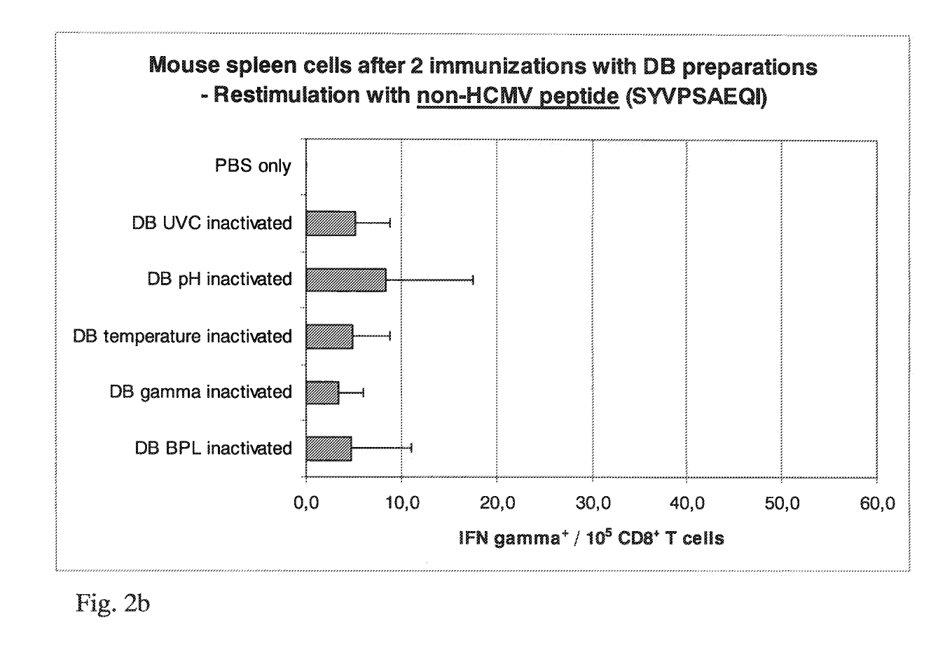

FIG. 2 shows CD8+ cytotoxic T cell response to DB preparations treated with various inactivation procedures upon restimulation with pp65 peptide mix (FIG. 2a) and with non-HCMV peptide (FIG. 2b);

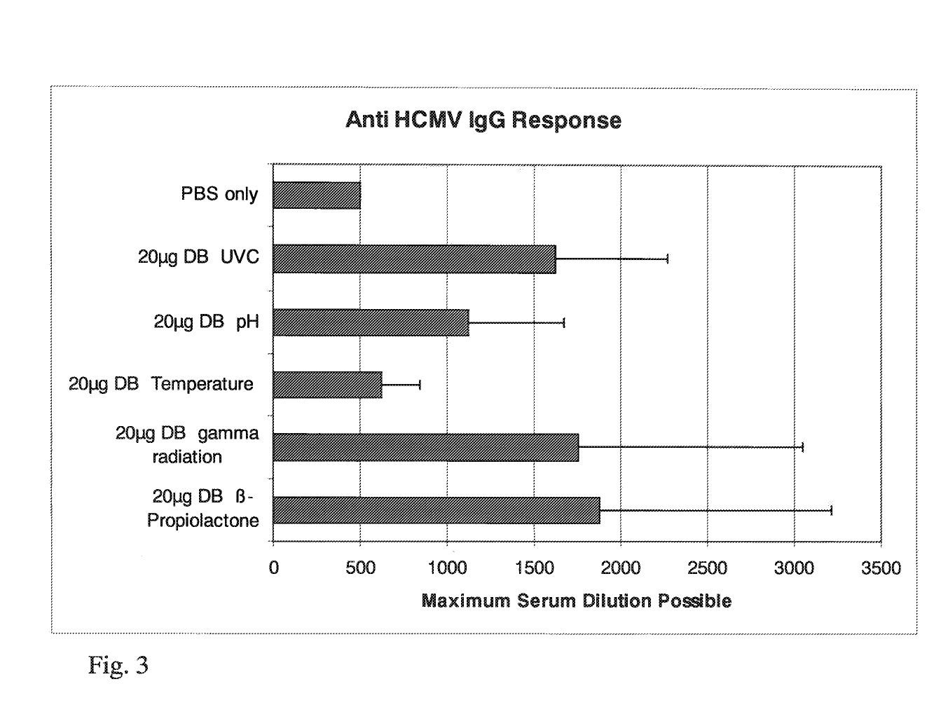

FIG. 3 shows anti HCMV IgG Response of DB preparations treated with various inactivation procedures;

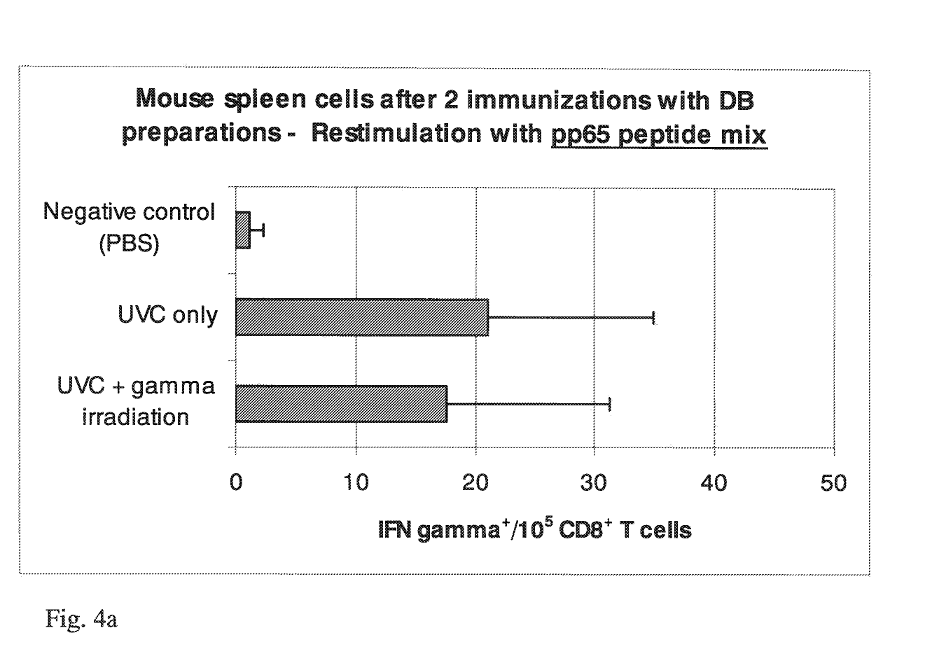

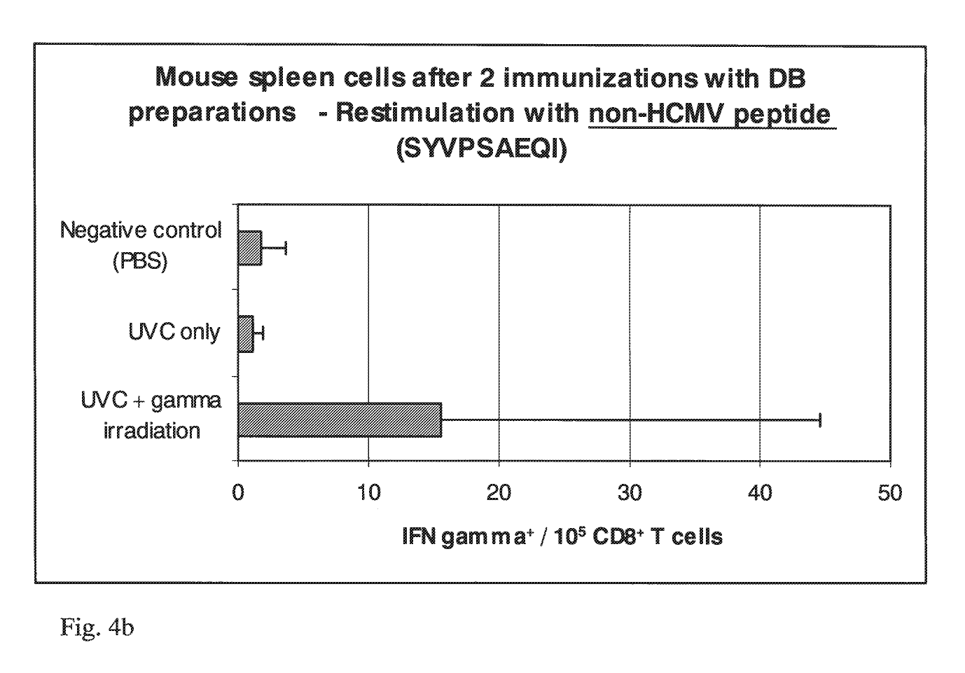

FIG. 4 shows CD8+ cytotoxic T cell response to DB preparations treated with two different inactivation procedures upon restimulation with pp65 peptide mix (FIG. 4a) and with non-HCMV peptide (FIG. 4b);

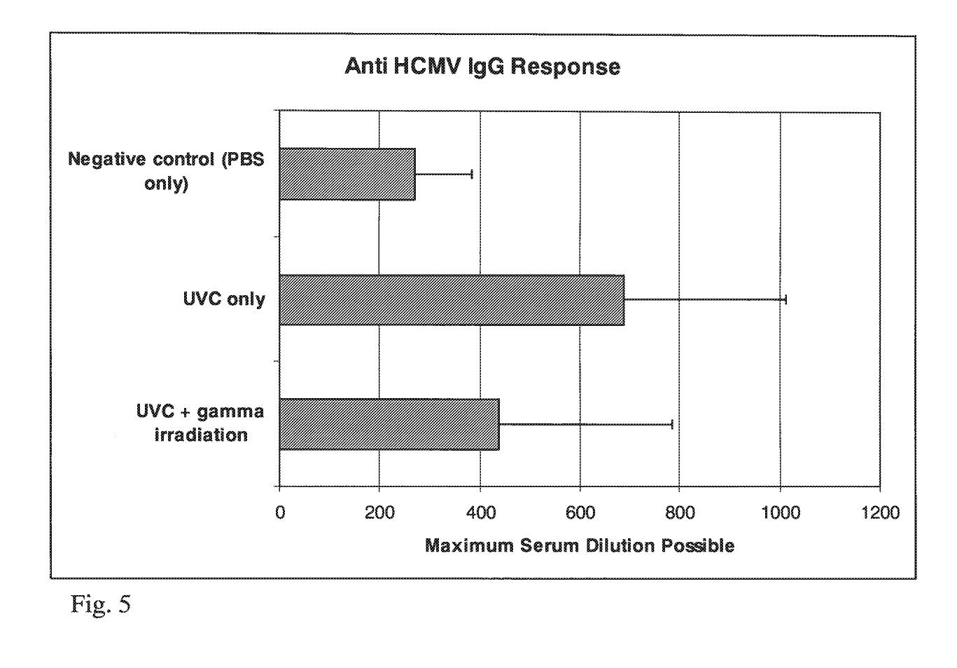

FIG. 5 shows anti HCMV IgG Response of DB preparations treated with two different inactivation procedures;

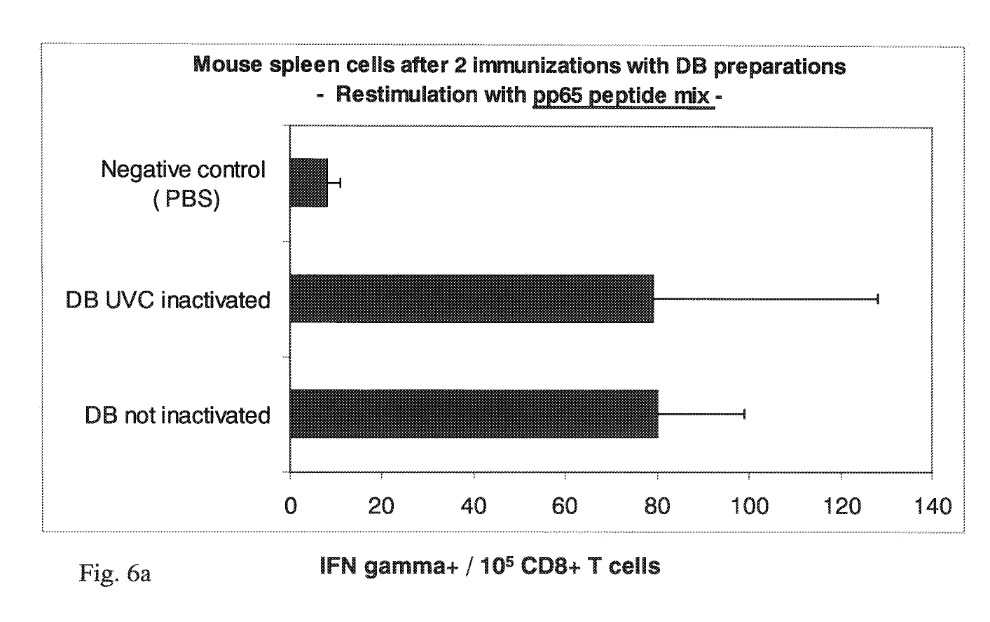

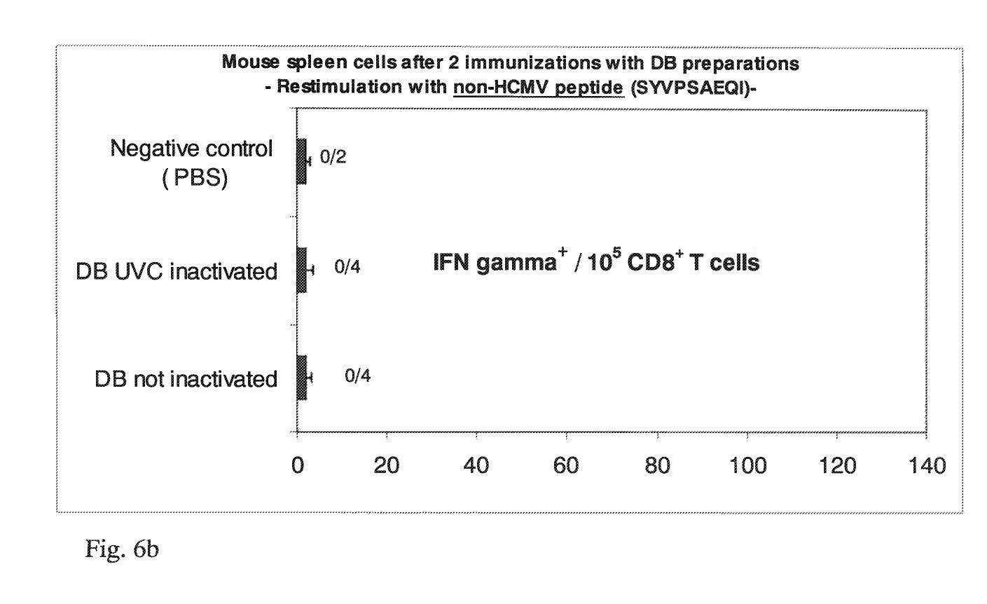

FIG. 6 shows CD8+ cytotoxic T cell response to DB preparations which were either treated to inactivate residual infectivity and to render them non-fusiogenic (semi dynamic UVC irradiation), or which did not receive such treatment. Restimulation with pp65 peptide mix (FIG. 6a) and with non-HCMV peptide (FIG. 6b);

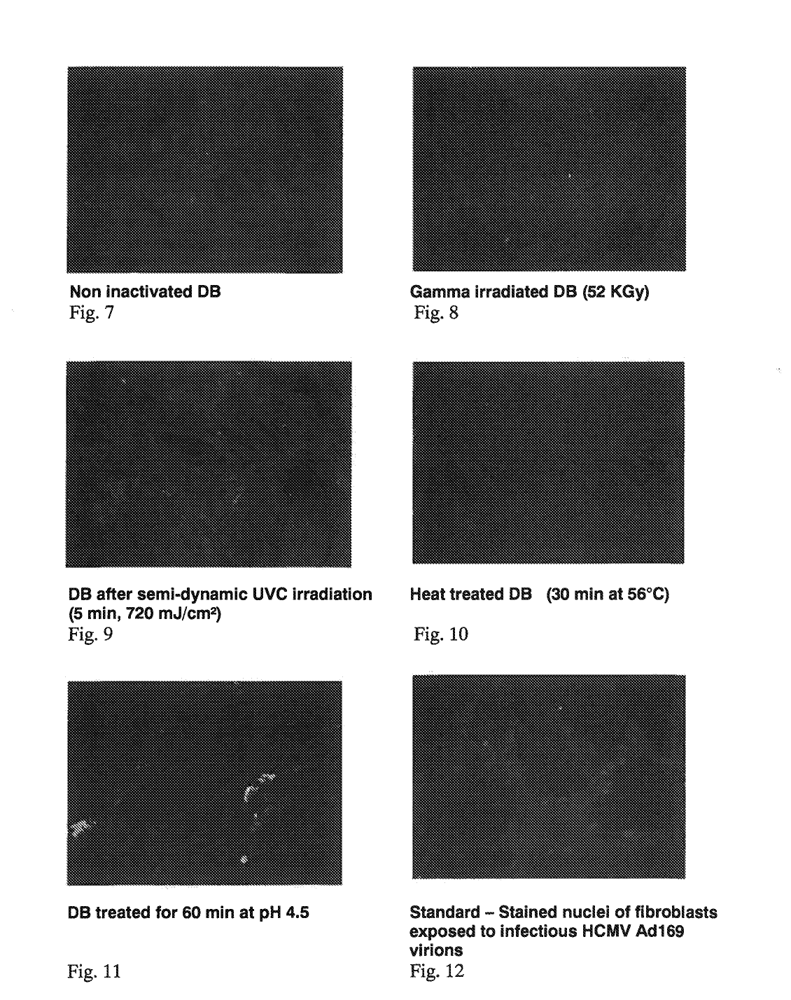

FIGS. 7 to 12 are microphotographs showing the result of a fusiogenicity assay, whereby the HCMV particles tested for fusiogenicity had been subjected to different inactivation methods; and

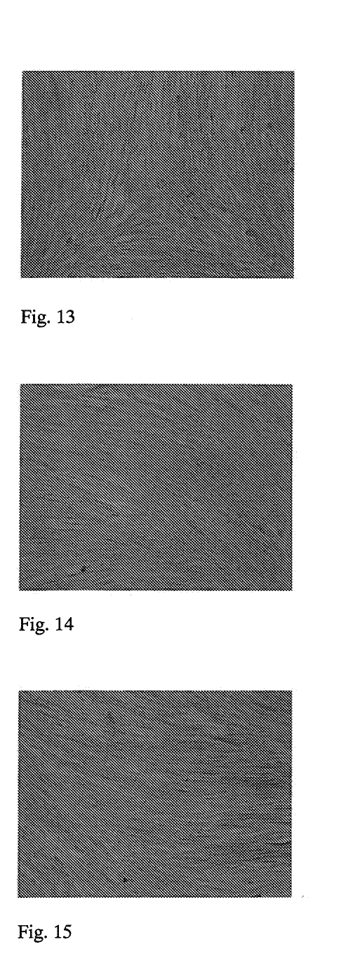

FIGS. 13 to 19 are microphotographs showing the result of an infectivity assay, whereby the HCMV particles tested for fusiogenicity had been subjected to different inactivation methods.

### EXAMPLE 1

### Materials and Methods

The following is an outline of the various materials and methods which have been used in the practicing of the present invention or which will be useful for a person skilled in the art practicing the present invention.

In brief, HCMV DB preparations received treatment to inactivate residual HCMV infectivity and to render the material non-fusiogenic. Afterwards, four different major types of analyses were done with the inactivated material. The ability to induce an antigen specific CD8+ cytotoxic T cell response in mice was analyzed. The ability to induce specific anti-HCMV antibodies was analyzed. The infectivity of DB material treated to inactivate residual infectivity was analyzed. The fusiogenicity of the inactivated material was analyzed.

### 1. Inactivation of Residual HCMV Infectivity

Various methods were used in connection with the present invention so as to inactivate residual HCMV infectivity in compositions containing HCMV virions and/or HCMV dense bodies.

### 1.1 Semi Dynamic UVC Treatment

300 μl aliquots of DB preparations (0.2 mg protein/ml PBS) were irradiated for 5 minutes in a slowly shaking 24-well cell culture plate with UVC light from above (254 nm; UVC dose of 720 mJ/cm²; UVC light: Schütt Osram HNS 11 Watt).

### 1.2 Gamma Irradiation

Aliquots of 675 μl of DB preparations (0.2 mg protein/ml) in 2 ml glass vials, received 52 KGy of gamma radiation on dry ice. After the inactivation process the material was frozen at −80° C. for subsequent analysis.

### 1.3 Low pH

1.15 ml of DB preparations (0.2 mg protein/ml) were mixed with 100 mM sodium citrate pH 4.5 and were incubated at 30° C. for 60 min. Afterwards material was pelleted by ultracentrifugation (45 min, SW 50.1 rotor) and resuspended in 1.1 ml PBS for subsequent analysis (Storage at −80° C.).

### 1.4 Heat Treatment

1.15 ml of DB preparations (0.2 mg protein/ml) were incubated at 56° C. for 30 min. After the inactivation process the material was frozen at −80° C. for subsequent analysis.

### 1.5 B-Propiolactone (BPL)

1.15 ml sample (0.2 mg protein/ml) were mixed with 10 ml of 50 mM Tris/HCl pH 8.0 and 0.24 ml of a freshly prepared 10% solution of BPL in 50 mM Tris/HCl pH 8.0 (0.21% v/v BPL final concentration). Samples were incubated for 60 min at 30° C. Afterwards material was pelleted by ultracentrifugation (45 min, SW 50.1 rotor) and resuspended in 1.1 ml PBS for subsequent analysis (Storage at −80° C.).

1.6 Dynamic UVC Treatment with Subsequent Gamma Irradiation

HCMV particle containing cell culture supernatant (medium without dye) received a dynamic UVC dose of 200 mJ/cm² with the small UVivatec Lab unit (supplied by BTS Bayer Technology Services, D-51368 Leverkusen, Germany). The dose at 254 nm was calculated according to the calculation sheet supplied by BTS. Subsequently the DB preparations were filled in glass vials and received 23.8 KGy of gamma irradiation on dry ice. Storage at −80° C. for subsequent analysis.

### 2. Testing the Ability of HCMV Virions and HCMV Dense Bodies to Induce an Antigen Specific CD8+ Cytotoxic T Cell Response in Mice

Overview: Mice will be immunized with DB material which has been treated to inactivate residual HCMV infectivity. Mice will be sacrificed and spleens will be removed. Subsequently spleen single cell suspensions will be prepared and red blood cells will be lysed. Afterwards the remaining spleen cells will be put in cell culture and incubated with defined peptides. Later on, spleen cells will be analyzed for the number of IFN-gamma positive CD8+ T cells. Peptides specific to the HCMV antigen pp65 will restimulate the spleen cells since they originate from mice immunized with pp65 contained in DB. In this assay CD8 positive cytotoxic T-cells (contained in the spleen cells suspension) that are specific for pp65 will produce IFN-gamma. Treatment of spleen cells with an irrelevant, non-HCMV peptide serves as a negative control. Treatment with an irrelevant, non-HCMV peptide should not lead to the induction of IFN-gamma positive CD8+ cytotoxic T cells since the mice were not immunized with this antigen before. The analysis of IFN-gamma producing CD8+ T cytotoxic cells will be performed by means of flow cytometry (FACS).

### 2.1 Immunization of Mice

8 week old female BALB/c mice were immunized (s.c.) with 20 μg of a DB preparation. After 3 weeks they received a boost (s.c.) with 20 μg of a DB preparation. After another 2 weeks animals were sacrificed for analysis of pp65-specific CTL response, for analysis of antigen-specific CD4+ T helper cells, for analysis of an HCMV-specific antibody response and for an analysis of an HCMV-neutralizing antibody response.
2.2 Re-Stimulation of pp65 Specific CTL in Mouse Spleen

### 2.2.1. Preparation of Spleen Cells

warm all needed buffers to 37° C.
take spleens
place 100 nm Falcon Nylon sieve onto 50 ml falcon tube, squeeze spleen through mesh to generate single cell suspension, rinse with total 10 ml PBS/1% FCS
centrifuge 1400 rpm (250-300 g), 5 Min, 20° C.
discard supernatant, resuspend pellet in 10 ml erythrocyte lysis buffer (37° C.)
incubate 3 Min at room temperature (RT)
centrifuge 1400 rpm (250-300 g), 4 Min, 20° C.
wash pellet with 10 ml PBS/1% FCS; centrifuge 1400 rpm, 4 Min, 20° C.; re-suspend second time with 10 ml PBS/1% FCS and remove clumps of connective tissue
take aliquot to determine cell concentration (use 1:10 dilution for determination)
Centrifuge 1400 rpm (250-300 g), 4 Min, 20° C.
Resuspend pellet in Click's RPMI/full media, adjusting concentration to 1.5×10⁷ cells/ml
4 parallel wells (100 μl cell suspension per well) per re-stimulation type needed (quadruplicates)

### 2.2.2 Peptides

2.2.2.1 For Re-Stimulation with HCMV Pp65 Specific Peptides

Reagent: PepMix pp65 HCMVA, from JPT Peptide Technologies GmbH, 10115 Berlin; pp65 (HCMVA) #P06725, 1 vial=25 μg per peptide; mix of 138 peptides (15mers, overlap 11); storage stock of 400 μg/ml in analytical grade DMSO (kept at −20° C.); prepare a working stock of 2 μg/ml.

2.2.2.2 For Re-Stimulation with Non-Relevant Control Peptide

Reagent: non-relevant nonamer control peptide. For example, for BALB/c mice a Kd binding malaria peptide (SYVPSAEQI); storage stock of 1 mg/ml in DMSO (kept at −20° C.); prepare a working stock of 2 μg/ml.

### 2.2.3 Brefeldin A

Prepare Brefeldin A stock (Sigma #B-7651) of 10 mg/ml in DMSO; prepare working solution of 20 μg/ml in Click's RPMI/full medium (5 μg/ml final concentration in re-stimulation well).

Brefeldin A blocks the golgi transport thereby inhibiting secretion of produced cytokines. cytokines remain intracellular under Brefeldin A treatment so that cytokine producing cells can be detected using intracellular staining methods.

### 2.2.4 PMA/Ionomycin

PMA (Phorbol 12-myristate 13-acetate), Sigma #P8139; prepare stock of 1 mg/ml in DMSO; prepare working solution of 0.4 μg/ml in Click's RPMI/full medium (0.05 μg PMA/ml in re-stimulation well).

Ionomycin, Sigma #10634; prepare stock of 1 mg/ml in DMSO; prepare working solution of 4 μg/ml in Click's RPMI/full medium (0.5 μg PMA/ml in re-stimulation well).

PMA and ionomycin polyclonally stimulate T cells. It serves as a positive control in the assay to be sure that cell quality and intracellular staining procedures were optimal.

### 2.2.5 Click's RPMI/Full Medium

10.81 g powder Click's RPMI (+L-Glutamine/−NaHCO₃) from AppliChem #A2504.

### +1.175 g NaHCO₃

Ad 1 l with destilled water→sterile filtration

### +Glutamine 2 mM

+PenStrep−100 U/ml Penicillin, 100 μg/ml Streptomycin
+5% fetal calf serum (FCS), Invitrogen/Gibco #10106-185,

### +β-Mercaptoethanol 4×10⁻⁶ M

+Hepes buffer 10 mM, Invitrogen/Gibco #15630-049,

### 2.2.6 Re-Stimulation Set Up

Per spleen and peptide used for re-stimulation pipet 100 μl spleen cell suspension in 4 wells of 96-round bottom plate, each (final 1,5×10⁶ cells per well)
add 50 μl peptide working solution or 25 μl PMA/25 μl Ionomycin working solution
add 50 μl Brefeldin A (with 20 μg/ml)
incubate 4 h at 37° C. (5% CO₂) before FACS analysis
   2.3 CD8 positive (CD8⁺) T cells/intracellular IFNγ staining for FACS analysis

Day 1:
adjust fixation buffer to room temperature (RT)
centrifuge 96-well round bottom plate containing the re-stimulation set up (1400 rpm/4 min/4° C./with brake; 250-300 g).
Pool pellets of parallel incubations (quadruplicates) with 90 μl buffer A (PBS+0.5% (w/v) BSA+0.1% (w/v) NaN₃) and transfer into new 96-well round bottom plate. Repeat rinsing with 90 μl Volume, to obtain all remaining cells from re-stimulation set up.→180 μl total volume per set up type.
Centrifuge new plate
add 120 μl Hybridoma supernatant (2.4G2) per well, mix and incubate 15 min at 4° C.
centrifuge plate+remove supernatant
add 100 μl anti-mouse CD8.PE (diluted 1:200 in buffer A)→re-suspend pellet→incubate 20 Min, 4° C.
after incubation time add 100 μl buffer A
centrifuge plate and wash two times with 150 μl Puffer A/centrifuge
add 150 μl fixation buffer (1% Paraformaldehyde in PBS) to the pellet, re-suspend→incubate 15 min at RT (in dark)
centrifuge plate re-suspend pellet with 150 μl buffer A
plate can be stored at 4° C. like this over one or two nights before proceeding with intracellular staining

### Day 2:

centrifuge plate→re-suspend with 150 μl buffer B per well incubate 15 min at RT (dark)
centrifuge add 50 μl/well anti-IFNγ.FITC, (1:200 in buffer B diluted)→re-suspend and incubate 30 min at RT (in dark)
add 100 μl buffer B (PBS+0.5% (w/v) BSA+0.5% (w/v) Saponin+0.05% (w/v) NaN₃), spin.
wash three times with 150 μl buffer B per well
re-suspend cells in 150 μl buffer A per well and transfer into FACS-tubes
re-suspend remaining cells again in 150 μl Puffer A and pool into same FACS tube
analyse 60.000 CD8+ T cells per sample by flow cytometry

### Other Reagents

Falcon Nylon-sieves 100 μm (Falcon Cat #352360).
Anti mouse CD8a PE conjugate, 0.2 mg/ml; Cat #553033; BD Biosciences.
Rat anti mouse IFNy FITC conjugated, 0.1 mg; rat IgG1; Clone XMG1.2, Cat #554411; BD Biosciences.
Paraformaldehyde EM Grade, Cat #00380-250; 250 mg Polysciences Europe.
Saponin (from Quillaja bark), Sigma #S-2149; 25 g;

### Erythrocyte Lysis Buffer

Prepare NH₄Cl stock solution 0.16 M. Prepare Tris stock solution 0.17 M. Mix 4.5 Liter NH₄Cl stock and 0.5 liter Tris stock, stir for 1 hour, then adjust to pH 7.2. Autoclave.

### 2.4G2 Hybridoma Supernatant

Anti-Fcγ Receptor FcRII; culture supernatant from about 4-day culture (dense cell lawn). Anti-Fcγ Receptor FcRII is to prevent nonspecific binding of antibodies used for staining of CD8 and IFNγ to cellular Fc receptors. Nonspecific binding of antibodies used for staining would lead to false positive signals.

The 2.4G2 hybridoma is available from ATCC.

### Fixation Buffer

1% Paraformaldehyde in PBS: 1 g paraformaldehyde in 100 ml PBS [weight under chemical hood!]; heat 1 hour at 70° C. to dissolve; store at 4° C.

3. ELISA to determine the anti HCMV IgG response in mice
Material: SERION ELISA classic CMV IgG (Clindia); strips coated with HCMV lysate, ready to use (ESR109G)
Sample: mouse serum (see 2.1)

### Procedure:

Serum Add 100 μl of diluted serum (in PBS/T) into each well e.g. 1:125; 1:250; 1:500; 1:1000; 1:2000; 1:4000 and 1:8000
   Incubate for 1 hour at 37° C. without shaking
Washing: Flick out the Antibody Solution into the sink.
   Wash plates by rinsing the wells 5× with 200 μl Washing Buffer each.
   After the third washing step, remove residual liquid by gently flicking the plate face down on a layer of several paper towels.
AK/HRP: Add AK anti-mouse IgG HRP conjugate diluted 1:1000 in PBS/T
   100 μl/well, incubate for 1 hour at 37° C. without shaking
Washing: Repeat step 2
Staining: Prepare staining solution just prior to use:
   1 mg OPD/mL Substrate buffer+1 μl/ml H₂O₂
   (e.g. dissolve 11 mg OPD/11 ml Substrate buffer, add 11 μl H₂O₂)
   Add staining liquid 100 μl/well, incubate at RT in the dark for 10 to 15′
Stopping Add 50 μl stop solution and measure in an ELISA reader at 492 nm.

### Reagents

PBST PBS with 0.05% (v/v) Tween 20
Substrate buffer 0.1 M KH₂PO₄ pH 6.0

### Stop Solution 1 NH₂SO₄ (=0.5 M H₂SO₄)

Substrate (OPD) O-Phenylenediamine, crystalline; Sigma P-2903
H₂O₂ 30%

Antibody polyclonal rabbit anti-mouse IgG HRP conjugate, DAKO (1.3 g/l),
#P0260

### 4. Testing the Fusiogenicity of HCMV Particles

The purpose of this assay is to test whether HCMV particles are still able to fuse with a particular target cell such as MRC5 human fibroblasts. It is analyzed whether the HCMV pp65 protein can be introduced into the respective target cells. This is done by immunofluorescence microscopy (anti pp65; green fluorescent staining in the nucleus). By fusion of the DB with the fibroblast, pp65 protein gets released into the cytoplasm of the target cell. As pp65 gets transported into the nucleus it is detected there by immunostaining. In HCMV particles that are not fused with HCMV-negative target cells, the tegument protein pp65 is located inside the HCMV particles. It is not present within the target cells. Vice versa, in cells which fused with HCMV particles, there will be a green fluorescent staining in the cells indicating the presence of pp65 in the nucleus of the cells.

### Protocol Fusiogenicity Assay:

Put 100 μl of MRC5 human fibroblast cells (ATCC, #ATCC-CCL-171) from an ongoing culture in fresh culture (1×10⁵ cells/ml culture medium; 37° C., 5% CO₂; quadruplicates; 96 well plates).
Incubate over night at 37° C.
Remove 70 μl medium from each well and add 5 μl of sample to be tested for fusiogenicity
After 1 h add back 70 μl of medium and incubate for 24 h
Remove supernatant from the cells (96 well plate)
Add 200 μl/well of 96% ethanol and incubate 20 min at RT
Wash 4 times with 150 μl PBS/0.1% Triton X100 per well
block with 50 μl of SN2.4G2 per well for 15 min at RT
remove supernatant from cells
Add primary antibody (50 Owen): anti-pp65, 1:100 dilution in PBS, #C8A023M.
Incubate for 1 h a 37° C.
Wash 3 times with 150 μl PBS/0.1% Triton X-100 per well.
50 μl/well: Add secondary antibody+Evans Blue (2.Ab 1:50/Evans Blue 1:25 in PBS), #E0413, and incubate for 30 min at 37° C.
Wash 4 times with 150 μl PBS/0.1% Triton X100 per well
Add 50 μl/well Streptavidin/FITC (1:100 in PBS), Beckman Coulter, #PN IM 0307.
Incubate for 15 min at 4° C.
Wash 3 times with 150 μl PBS/0.1% Triton X100 per well.
Add 150 μl PBS per well (without TX100) and store at 4° C., wrapped with aluminum foil (protected from light) until ready for analysis by fluorescence microscopy.
Analyze by means of fluorescence microscopy.

### Primary Antibody:

To show fusiogenicity: anti pp65, clone 1-L-11, mouse ascites,
Biodesign, Cat#C8A023M, 1 mg/ml; use diluted 1:100 in PBS.

### Secondary Antibody:

Polyclonal rabbit anti-mouse Ig/Biotinylated Rabbit F(ab′)2
Dako, #E0413, (0.79 g/l); use diluted 1:50 in PBS.

### Evans Blue:

Fluka #46160—dissolve to 0.5% in PBS and use diluted 1:25 in PBS.

### Streptavidin-DTAF (Strep/FITC)

Beckman Coulter, #PN IM 0307 (1.8 mg/ml); use diluted 1:100 in PBS.
   PBS/0.1% Triton X-100 (to wash).
   SN2.4G2 Hybridoma supernatant
   Anti-Fcγ Rezeptor FcRII; culture supernatant from about 4-day culture (dense cell lawn).
   Anti-Fcγ Receptor FcRII is to prevent nonspecific binding of antibodies used for staining of CD8 and IFNγ to cellular Fc receptors. Nonspecific binding of antibodies used for staining would lead to false positive signals.
   The 2.4G2 hybridoma is available from ATCC.

### Medium:

MEM with 10% FCS

### 2 mM Glutamine

50 mg/ml Gentamycin

### 1 mM MEM Sodiumpyruvat

1×NEAA (nonessential aminoacids)

### 5. Testing the Infectivity of HCMV Particles

The infectivity testing assay is used to ascertain an effective virus inactivation. In this assay fibroblast cells are incubated either together with infectious (positive control) or inactivated (non-infectious) virus containing samples. The subsequent AEC (=3-Amino-9-ethylcarbazole) staining is an immuno-histochemistry assay visualizing target proteins. In this case, the monoclonal mouse antibody is targeted against HCMV IEA (immediate early antigen), a viral protein that appears shortly after cell infection IEA reaches the intensity peak at 48 hours and persists during the entire HCMV infection cycle. The secondary antibody is an anti-mouse polyclonal antibody conjugated to HRP (horseradish peroxidase). Unbound conjugate is washed off and a chromogenic substrate (AEC) is added. This substrate is hydrolyzed by the bound enzyme conjugate and produces an insoluble end product that is red in colour and can be observed visually by microscopy. Since IEA is a nuclear protein, cells that have been infected with HCMV can be identified by their coloured nuclei. The reference item only serves as a control for the staining procedure. A given sample is regarded as non-infectious when no cell nuclei are stained in the respective wells.

### Protocol for Infectivity Assay

100 μl of MRC5 human fibroblast cells (ATCC, #CCL-171) from an ongoing culture in fresh culture (1×10⁵ cells/ml culture medium; 37° C., 5% CO₂; quadruplicates; 96 well plates).
After 24 hours add 100 μl of a dilution of material to be tested for infectivity to 100 μl of cells (e.g., in Tab. 2: A 1:200 dilution of reference item or 0.3 μg protein of test items, respectively)
Incubate for 48 h at 37° C.
Remove HCMV supernatant after 48 h
Wash cells with 150 μl PBS/well, each
Fix the cells with 96% ethanol (200 μl/well) for 20 min at RT
Wash 2 times with PBS (150 μl/well)
Add primary antibody directed to IE antigen (αHCMV IEA, Argene; #11-003), diluted 1:100 in PBS (50 μl/well)
Incubate for 60 min at 37° C.; wrapped with plastic wrap in a moist chamber (incubator)
Wash 3× with PBS/0.1% Triton X100, 150 μl/well, each.
Add secondary antibody: Anti-Mouse Peroxidase (e.g., Dako P0260) diluted 1:500 in PBS (50 Ξl/well)
Incubate for 60 min at 37° C.; wrapped with plastic wrap in a moist chamber (incubator)
Wash 3× with PBS/0.1% Triton X100, 150 μl/well, each.
Staining: AEC-Stock diluted 1:20 in acetate buffer, 2× filtered through paper filter which were prewetted with acetate buffer before.
Just before the staining procedure add 1:1000 H₂O₂ (30%)
From this staining solution add 100 μl/well
Incubate for exactly 1 h at 37° C. in the dark (incubator)
Wash with 2×PBS, 150 μl/well, each
For microscopy add 1×PBS (150 μl/well); store at 4° C. Infected nuclei will be bright red.
   AEC stock:
400 mg AEC (=3-Amino-9-ethylcarbazole; Sigma; #A6926) make up to 100 ml with DMF (Dimethyl-Formamid; Roth; #6251.1); prepare 2 ml aliquots and store at −20° C.
   Acetate buffer:
   13.6 g sodium acetate×3 H₂O+2.88 ml glacial acetic acid+H₂O to 1000 ml adjust pH 4.9.

### Medium:

MEM with 10% FCS

### 2 mM Glutamine

50 mg/ml Gentamycin
1 mM MEM sodium pyruvate
1×NEAA (nonessential amino acids)

### EXAMPLE 2

### Results

Results of pp65 Specific CD8+ CTL Response

In summary, this shows, that DB preparations treated to inactivate residual HCMV infectivity and to render DB preparations non-fusiogenic remained able to induce a pp65 specific CD8+ cytotoxic T cell response in mice (FIGS. 2 and 4) Treated samples were non-fusiogenic as depicted in FIGS. 7 to 12 and, as shown in Tab. 2, were non-infectious. DB preparations treated to inactivate residual HCMV infectivity and to render DB preparations non-fusiogenic were shown to be equally immunogenic as non-treated HCMV preparations (FIG. 6). This was judged by HCMV pp65-specific CD8+ cytotoxic T cell response in mice.

FIG. 2 shows results from Balb/C mice that were immunized with DB material which was treated to inactivate residual infectivity and to render it non-fusiogenic as outlined in Example 1: UVC (720 mJ/cm²), low pH, high temperature, gamma irradiation (52 KGy) and β-propiolactone, respectively. The X-axis indicates the number of IFN-gamma producing CD8+ cytotoxic T cells per 10⁵ CD8+ T cells. In this assay CD8 positive cytotoxic T-cells (contained in the spleen cells suspension) that are specific for pp65 will produce IFN-gamma (FIG. 2a). Treatment of spleen cells with an irrelevant, non-HCMV peptide serves as a negative control for the restimulation (FIG. 2b). The results show, that even DB preparations treated to inactivate residual infectivity are still able to induce a CD8+ cytotoxic T cell response specific to HCMV pp65. Mice immunized with PBS hardly indicated any IFN-gamma producing CD8+ T cells.

FIG. 4 shows results from Balb/C mice that were immunized with DB material which was treated to inactivate residual infectivity and to render it non-fusiogenic as outlined in Example 1: either UVC irradiation only or dynamic UVC and subsequent gamma irradiation (23.8 KGy). The X-axis indicates the number of IFN-gamma producing CD8+ cytotoxic T cells per 10⁵ CD8+ T cells. In this assay CD8 positive cytotoxic T-cells (contained in the spleen cells suspension) that are specific for pp65 will produce IFN-gamma (FIG. 4a). Treatment of spleen cells with an irrelevant, non-HCMV peptide serves as a negative control for the restimulation (FIG. 4b). The results show, that even DB preparations treated with a combination of two irradiation procedures are still able to induce a CD8+ cytotoxic T cell response specific to HCMV pp65. Mice immunized with PBS hardly indicated any IFN-gamma producing CD8+ T cells.

FIG. 6 shows results from Balb/C mice that were immunized with DB material. The material was either treated to inactivate residual infectivity and to render it non-fusiogenic (semi dynamic UVC irradiation), or the material did not receive such treatment.

The X-axis indicates the number of IFN-gamma producing CD8+ cytotoxic T cells per 10⁵ CD8+ T cells. In this assay CD8 positive cytotoxic T-cells (contained in the spleen cells suspension) that are specific for pp65 will produce IFN-gamma (FIG. 6a). Treatment of spleen cells with an irrelevant, non-HCMV peptide serves as a negative control for the restimulation (FIG. 6b). The results show, that DB preparations treated to inactivate residual HCMV infectivity and to render DB preparations non-fusiogenic were equally immunogenic as non-treated HCMV preparations. This was judged by HCMV pp65-specific CD8+ cytotoxic T cell response in mice. Mice immunized with PBS hardly indicated any IFN-gamma producing CD8+ T cells.

Results of anti HCMV IgG Response

In summary, this shows, that DB preparations treated to inactivate residual HCMV infectivity and to render DB preparations non-fusiogenic remained able to induce a specific anti-HCMV IgG response (FIGS. 3 and 5). The samples were non-fusiogenic as indicated in FIGS. 7 to 12 and non-infectious as shown in Tab. 2.

FIG. 3 shows results from Balb/C mice that were immunized with DB material which was treated to inactivate residual infectivity and to render it non-fusiogenic as outlined in Example 1: UVC (720 mJ/cm²), low pH, high temperature, gamma irradiation (52KGy) and β-propiolactone, respectively. The higher the maximum serum dilution that is still able to induce an assay signal, the stronger the anti-HCMV response induced. The results show, that even DB preparations treated with a combination of two irradiation procedures are still able to induce a specific anti-HCMV IgG response.

FIG. 5 shows results from Balb/C mice that were immunized with DB material which was treated to inactivate residual infectivity and to render it non-fusiogenic as outlined in Example 1: either UVC irradiation only or dynamic UVC and subsequent gamma irradiation (23.8 KGy). The higher the maximum serum dilution that is still able to induce an assay signal, the stronger the anti-HCMV response induced. The results show, that even DB preparations treated with a combination of two irradiation procedures are still able to induce a specific anti-HCMV IgG response.

### Results Fusiogenicity Assay:

In summary, results show, that the inactivated DB preparations used to demonstrate a pp65 specific CD8+ cytotoxic T cell response (FIGS. 2 and 4) as well as induction of specific anti-HCMV antibodies (FIGS. 3 and 5) were non-fusiogenic; and, as shown in Tab. 2, were non-infectious. Samples were treated as outlined in Example 1. The non-specific green staining in sample P4 was due to an artefact.

### Result of a Typical Infectivity Assay

In summary, results show, that DB preparations treated to inactivate residual HCMV infectivity and to render DB preparations non-fusiogenic were non-infectious. However, these samples remained immunogenic as judged by their ability to induce a CD8 positive cytotoxic T cell response specific to the HCMV antigen pp65 (FIGS. 2 and 4) and as judged by their ability to induce specific anti-HCMV IgG (FIGS. 3 and 5). Cells incubated only with medium served as negative control. Only the non-inactivated reference item was able to induce red stained nuclei, i.e. was infectious. Samples were treated as outlined in Example 1.

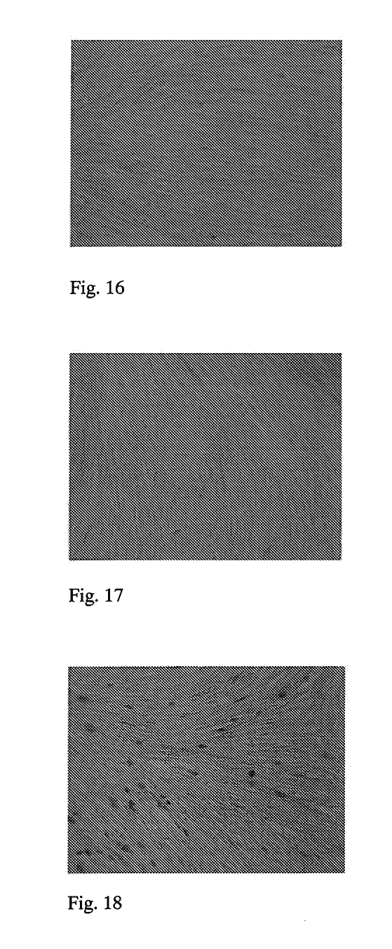

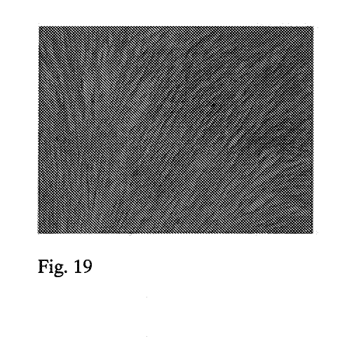

TABLE 2

| | | Picture | |
|---|---|---|---|
| | Amount of | documentation of | Infectious |
| | protein used | the respective | HCMV |
| | or initial | stainings of cell | particles/ |
| Sample type | dilution | nuclei depicted in | ml |
| Semi dynamic UVC | 0.3 μg protein | FIG. 13 | 0 |
| irradiation | | | |
| (5 min, 720 mJ/cm²) | | | |
| pH 4.5 for 60 min at | 0.3 μg protein | FIG. 14 | 0 |
| 30° C. | | | |
| 30 min at 56° C. | 0.3 μg protein | FIG. 15 | 0 |
| gamma-irradiation | 0.3 μg protein | FIG. 16 | 0 |
| (52 KGy) | | | |
| β-Propiolactone | 0.3 μg protein | FIG. 17 | 0 |
| (0.21% BPL, 60 min | | | |
| at 30° C.) | | | |
| Dynamic UVC (200 | 0.3 μg protein | No picture taken; | 0 |
| mJ/cm²) and gamma- | | result was | |
| irradiation (23.8 | | documented | |
| KGy) | | | |
| Reference item: | 1:200 | FIG. 18 | 6.82e6 |
| non-inactivated | | | |
| HCMV virions Ad169 | | | |
| Medium only | N/A | FIG. 19 | 0 |

The features of the present invention disclosed in the specification, the claims and/or the drawings may both separately and in any combination thereof be material for realizing the invention in various forms thereof.

1. A method of elucidating an immune response against at least one antigen of HCMV in a patient in need thereof, comprising administering to said patient an effective amount of a composition comprising an agent selected from the group consisting of HCMV virions, HCMV dense bodies and HCMV NIEP, wherein the composition is capable of elucidating an immune response and wherein the virions, the NIEP and/or the dense bodies are non-fusiogenic.


SEQUENCE LISTING (1 sequence)

SEQ ID NO: 1 - protein, 9 aa, Artificial sequence
  Kd binding malaria peptide
SYVPSAEQI